(19) United States
(12) Patent Application Publication
(10) Pub. No.: US 2024/0130407 A1
(43) Pub. Date: Apr. 25, 2024

(54) COATED MEAT OR FISH SUBSTITUTE

(71) Applicant: DSM IP Assets B.V., HEERLEN (NL)

(72) Inventors: Frits BROUWER, ECHT (NL); Petrus Paulus Gerardus Anna VERHOEVEN, ECHT (NL)

(21) Appl. No.: 18/546,604

(22) PCT Filed: Feb. 18, 2022
(86) PCT No.: PCT/EP2022/054089

§ 371 Date: Aug. 15, 2023

(30) Foreign Application Priority Data
Feb. 22, 2021 (EP) 21158355.4

(51) Int. Cl. A23L 27/21; A23J 3/16; A23J 3/22; A23L 27/20; A23P 20/10

(52) U.S. Cl. CPC A23L 27/215; A23J 3/16; A23J 3/227; A23L 27/2052; A23L 27/2056; A23P 20/11

(57) ABSTRACT

The present invention relates to a meat substitute provided with a flavour agent coating, optionally wherein the weight of the flavour agent coating is between 0.1% and 5% of the weight of the coated meat substitute.

### FIELD

The present invention relates to a meat substitute. According to another aspect the present invention relates to a flavour agent coating composition. Finally, the present invention relates to a method for manufacturing a meat substitute.

### BACKGROUND OF THE INVENTION

Meat substitutes become abundantly available following the increasing need in the market for vegetarian products. Meat substitute hamburgers are developed that closely mimic the texture and taste of real meat hamburgers. Plant protein is the common ingredient in most meat substitute hamburgers. Challenges of using plant protein instead of animal protein is to provide a good flavour that is perceived as a real meat hamburger, without off flavours of the plant protein.

U.S. Ser. No. 10/172,380 relates to ground meat replicas and comprises about 0.00001% to about 10% by weight of a flavouring agent. The flavouring agent can include one or more flavour precursors, a flavouring, or a flavouring compound. The meat dough can include the flavouring agent or the fat can include the flavouring agent.

WO2021/009043 relates to ground meat analogue product comprising a plant protein extrudate, a plant protein dispersion and a fat mimetic. The meat analogue product may comprise flavourings, preferably meat flavourings. The flavourings are introduced into an extruder to include the flavouring into the dough.

The disadvantage is that meat analogue products require a high amount of flavour to achieve a pleasant meaty taste. Thus, adding of the flavour agent is not efficient and does not deliver the distinct fried meat flavour one would get from a real meat product fried in pan.

### DETAILED DESCRIPTION OF THE INVENTION

This object, amongst other objects is solved by providing a meat substitute according to the appended claims.

More specifically, this objective, amongst other objectives, is solved by providing a meat substitute provided with a flavour agent coating, optionally wherein the weight of the flavour agent coating is between 0.1% and 5% of the weight of the coated meat substitute.

The term “meat substitute” or “meat analogue” as used herein means a meat replacer which does not comprise animal material, like animal protein. Preferably, the meat substitute is suitable for consumption by a vegetarian or vegan consumer, optionally after cooking an/or heating.

The term “flavour agent” as used herein is an agent that is not intended to be consumed as such, but which are added to food in order to impart or modify odour and/or taste of the food item. Flavouring agents may comprise flavourings, flavour precursors and/or flavouring compounds.

The term “flavour precursor” as used in the present context means a product, not necessarily having flavouring properties itself, intentionally added to food for the sole purpose of producing flavour by breaking down or reacting (with other components) during food processing.

The term “provided with a flavour agent coating” as used herein means that the surface of the meat substitute is at least partly covered with the flavour agent coating. Preferably, the meat substitute is covered with the flavour agent coating for at least 30% of the substitute surface, more preferably at least 40%, at least 50%, at least 60%, at least 70%, at least 80%, at least 90%, at least 95% or even 100% of the substitute surface.

The present inventors found that providing a meat substitute with a flavour agent coating results in a meat substitute having meaty flavour impact after preparing the meat substitute for consumption.

Preferably, the present meat substitute is a raw, or uncooked, meat substitute. A raw or uncooked meat substitute means that the product needs a cooking step before consumption thereof.

In an embodiment, the meat substitute is a meat substitute hamburger. Alternatively, the meat substitute is chosen from the group consisting of nuggets, chicken nuggets, chicken chunks, pork pieces, tuna pieces, meat balls, fish fingers and fish sticks.

The term “meat substitute hamburger” as used herein means non-meat based flattened, usually round shape serving of ground meat substitute ingredients. Alternatively the present meat substitute hamburger is meat substitute patty, or meat substitute hamburger patty. The term hamburger is not intended to include the bun, or sandwich, and other toppings in which the present meat substitute hamburger is commonly served.

Preferably, the present meat substitute hamburger is at least coated on the one or two flat surfaces of the hamburger. Preferably, the present meat substitute hamburger is provided with a flavour agent coating at the entire surface of the hamburger. The entire surface includes the two flat surfaces of the hamburger as well the rim of the meat substitute hamburger. Such a coating can efficiently be applied by dipping the hamburgers in a coating bath.

The present inventors found that the coated meat substitute hamburger according to the invention has a good meaty flavour profile. This is a surprise since it shows that for providing a meaty flavour it suffices to provide the meat substitute hamburger with a flavour agent coating. Hence, the present invention provides an efficient use of flavour agent. This also allows a sufficient Maillard reaction in the short frying time of the meat substitute hamburger. This is advantageous in that the delicate plant based matrix of meat substitute hamburgers can not withstand high frying temperatures and suffer from degradation as a result of the applied heat. This can be avoided by shorter or less temperature frying, wherein the present coating still provides sufficient meaty flavour generation.

In an embodiment, the weight of the flavour agent coating is between 1.0 and 5%, preferably between 1.5% and 4% of the weight of the coated meat substitute, preferably between 2.0% to 3.5%, between 2.5% to 3.0% of the weight of the coated meat substitute.

In an embodiment, the flavour agent coating comprises flavour precursors. Examples of flavour precursors are reducing sugars (glucose, ribose, xylose, arabinose, fructose) nucleotides (inosine monophosphate (IMP) and guanosine monophosphate (GMP)), free amino acids (lysine, leucine, proline, glutamine) and peptides (GSH, GSSG, or glutathione peptides). Preferably the flavour precursors comprise cysteine, thiamine, glucose, ribose, xylose, methionine and/or cystine. The present inventors found that providing a meat substitute hamburger with a coating of flavour precursors provides a meat substitute hamburger with meaty impact. Without wishing to be bound by any theory, it is expected that the flavour precursors on the exterior of the hamburger are readily available for the Maillard reaction.

In another embodiment, the flavour agent coating comprises yeast extracts. According to the Food Chemical Codex “yeast extract” is defined as follows: “Yeast Extract comprises the water soluble components of the yeast cell, the composition of which is primarily amino-acids, peptides, carbohydrates and salts. Yeast Extract is produced through the hydrolysis of peptide bonds by the naturally occurring enzymes present in edible yeast or by the addition of food-grade enzymes”. The present inventors found that providing a coating comprising yeast extracts results in an improved meaty flavour impact, as well as a reduction of off taste of plant protein. This is surprising because the yeast extract contributes to flavour generation on the exterior of the meat substitute, but is able to efficiently flavour the whole meat substitute through and through, while reducing the off flavour of the plant protein that is present homogenously in the meat substitute. Further, it was found that a coating comprising yeast extracts provides a thicker crust on the meat substitute, which increases the meat like perception of the meat substitute. Without wishing to be bound by any theory, it is expected that yeast extracts provide a further improved meaty impact in view of flavour precursors because the amino acids in the yeast extract become more gradually available during the cooking reaction, whereas flavour precursors might be too reactive resulting in a loss of the flavour precursor. Further, a thicker crust improves retention of moisturizing components as water and fats inside the meat substitute, thereby contributing to the juiciness perception by the consumer. Preferably the present yeast extract comprises the present flavour precursors.

In a preferred embodiment, the present yeast extracts comprise an amount of reducing sugar of 1% to 30% wt. of the yeast extracts. Preferably, the amount of reducing sugar is 3% to 20% wt., preferably 4% to 15% wt. preferably 5% to 12% wt. of the yeast extracts. Preferably the reducing sugar is glucose. Glucose constitutes a widely accepted food component and is an effective Maillard agent. Preferably the reducing sugar is yeast own reducing sugar, or is derived from the oligosaccharides and polysaccharides that are normally present in the yeast cell. The advantage of using yeast derived reducing sugar is that it provides a more natural labelled meat substitute.

In a preferred embodiment, the present yeast extracts comprise an amount of free asparagine, based on dry matter, which is not higher than 1 mg/g, preferably not higher than 0.2 mg/g, more preferably not higher than 0.1 mg/g of the yeast extracts. The advantage of yeast extracts having a low amount of asparagine is that no or less acrylamide is formed during the Maillard reaction when preparing the meat substitute hamburger. Particularly if the yeast extract is used on the exterior of the meat substitute hamburger, there where the temperature is highest during preparing the hamburger, it is advantageous to use a low asparagine yeast extract.

Advantageously, the present meat substitute comprises an amount of acrylamide, based on product dry matter, of lower than 800 ppb, preferably lower than 600 ppb, preferably lower than 400 ppb, preferably lower than 200 ppb. Other preferred ranges are from 10 to 800 ppb, such as from 20 to 600 ppb, 50 to 400 ppb, or 100 to 200 ppb. Preferably after preparing the meat substitute by the consumer, the present flavour agent coating results in a crust having an amount of acrylamide, based on product dry matter of the crust, of lower than 800 ppb, preferably lower than 600 ppb, preferably lower than 400 ppb, preferably lower than 200 ppb. Other preferred ranges are from 10 to 800 ppb, such as from 20 to 600 ppb, 50 to 400 ppb, or 100 to 200 ppb.

In an embodiment, the yeast extracts comprise an amount of gluten of less than 100 ppm, preferably less than 50 ppm gluten, more preferably less than 40 ppm gluten, more preferably less than 30 ppm gluten, based on salt free dry matter of the yeast extract. This is advantageous in that the low gluten yeast extracts provide a meat substitute hamburger having an improved food label, without the necessity to mention the amount of gluten on the product.

Therefore, the present meat substitute comprises preferably an amount of gluten of less than 100 ppm, preferably less than 50 ppm gluten, more preferably less than 40 ppm gluten, more preferably less than 30 ppm gluten, based on dry matter of the meat substitute.

In an embodiment, the present flavour agent coating comprises an oil, preferably a plant oil. The advantage of using an oil is that the flavour agent is embedded in the oil matrix that glues the flavouring agent to the meat substitute. The further advantage of oil in the coating is that the present meat substitute can be prepared by the end user without adding oil to the pan. Examples of oil that can be used are chosen from the group consisting of arachis oil, sunflower oil, canola oil, flax seed oil, corn oil, soybean oil, palm oil, safflower oil, rice bran oil, cottonseed oil and olive oil.

Alternatively, the present meat substitute can be coated with a dry flavour agent coating. A dry flavour agent coating may comprise flour, egg white and/or crumbles. A dry flavour agent coating is advantageous for nugget or sticks that are normally provided with crumbles on the exterior as it mimics these conventional products.

In an embodiment, the present flavour agent coating comprises a reducing sugar or reducing sugar precursor. Preferably, the reducing sugar is chosen from the group consisting of galactose, glucose, glyceraldehyde, fructose, ribose, xylose, cellobiose, lactose, maltose, dextrin and maltodextrin.

In an embodiment, the present meat substitute hamburger comprises texturized vegetable protein, water, a binder and plant oil. The texturized plant protein is preferably soy texturized plant protein. Alternatively, the present texturized protein is texturized pea protein, texturized canola protein, texturized fava bean protein, texturized lupin protein, or combinations thereof. The plant oil can be chosen from the group consisting of arachis oil, sunflower oil, canola oil, flax seed oil, corn oil, soybean oil, palm oil, safflower oil, rice bran oil, cottonseed oil and olive oil. The binder can be methylcellulose, RuBisCO, albumin, egg white, gluten, calcium-alginate, starch and/or hydrocolloids.

In an embodiment, the present meat substitute has a weight within the range of 10 to 250 gram, preferably 50 to 200 gram, more preferably 70 to 180 gram, most preferably 90 to 160 gram. These weight ranges are advantageous in that the present inventors found that coatings with the range of 1% to 5% of the weight of the coated meat substitute are able to provide a meat flavour and reduce plant protein related off flavours through the entire meat substitute.

In an embodiment, the present meat substitute hamburger comprises flavouring and colorants. An example of flavouring can be heme containing protein, for providing a metallic taste to the meat substitute. This can advantageously be combined with a flavour agent coating comprising flavour precursors, to provide a roasty Maillard reacted flavour on the exterior of the meat substitute. In such a product, the hamburger matrix is provided with the desired flavour, whereas the present coating provides a roasty flavour.

The present colorant can be beetroot or comprises a carotenoid. Preferably, the carotenoid is chosen from the group consisting of α- or β-carotene, 8-′apo-β-carotenal, 8′-apo-β-carotenoic acid esters such as the ethyl ester, canthaxanthin, astaxanthin, astaxanthin esters, lycopene, lutein, zeaxan-thin or crocetin and their derivatives.

Preferably, the present meat substitute comprises a nutrient. Preferably, the nutrient comprises vitamins and/or minerals, preferably vitamins chosen from the group consisting of B2, B3, B6 and B12 and/or preferably minerals chosen from the group consisting of iron, selenium and zinc. The advantage of adding these nutrients is that the present meat substitute more closely resembles the nutritional value of a real meat product, without introducing off flavours to the meat substitute.

In a preferred embodiment the present meat substitute comprises a flavour or flavour agent, or flavour precursor. Examples of flavours can be yeast extracts or process flavours. More preferably the present meat substitute comprises 0.001 to 5 wt % of a flavour. The present inventors found that a meat substitute hamburger comprising a flavour in the dough (or in the interior, or in the matrix) as well as a flavour agent coating as provided herein results in a meat substitute hamburger having an improved meaty flavour profile.

Preferably, the present meat substitute comprises salt, preferable NaCl. The amount of salt is preferably within the range of 0.001 to 5 wt % of the present meat substitute.

According to another aspect, the present invention relates to a flavour agent coating composition comprising:
(i) 20% to 70% wt. of an oil;
(ii) 20% to 80% wt. of a yeast extract;
wherein the sum of (i) and (ii) does not exceed 100%.

The present oil can advantageously be used as a coating on the exterior of meat substitute hamburgers.

Preferably, the amount of oil is 30% to 60% wt, preferably 35% to 55% wt. of the flavour agent coating composition. The amount of yeast extract is preferably 30% to 70% wt., preferably 40% to 60% wt. of the flavour agent coating composition.

The oil is preferably plant oil, such as an oil chosen from the group consisting of arachis oil, sunflower oil, canola oil, flax seed oil, corn oil, soybean oil, palm oil, safflower oil, rice bran oil, cottonseed oil and olive oil.

Preferably, the yeast extract comprises an amount of free asparagine, based on dry matter, which is not higher than 1 mg/g, preferably not higher than 0.2 mg/g, more preferably not higher than 0.1 mg/g of the yeast extracts.

Preferably, the yeast extract comprises an amount of gluten of less than 100 ppm, preferably less than 50 ppm gluten, more preferably less than 40 ppm gluten, more preferably less than 30 ppm gluten, based on salt free dry matter of the yeast extract.

Preferably, the yeast extract comprises an amount of reducing sugar of 1% to 30% wt. of the yeast extract. Preferably, the amount of reducing sugar is 3% to 20% wt., preferably 4% to 15% wt. preferably 5% to 12% wt. of the yeast extracts. Preferably the reducing sugar is glucose or ribose.

In an embodiment, the present flavour agent coating composition comprises a reducing sugar or reducing sugar precursor. Preferably, the reducing sugar is chosen from the group consisting of galactose, glucose, glyceraldehyde, fructose, ribose, xylose, cellobiose, lactose, maltose, dextrin and maltodextrin.

In another aspect, the present invention relates to a method for manufacturing a meat substitute as defined above, comprising a step of coating a meat substitute hamburger with a flavour agent coating composition. Preferably a step of coating a meat substitute hamburger with a flavour agent coating composition as defined herein.

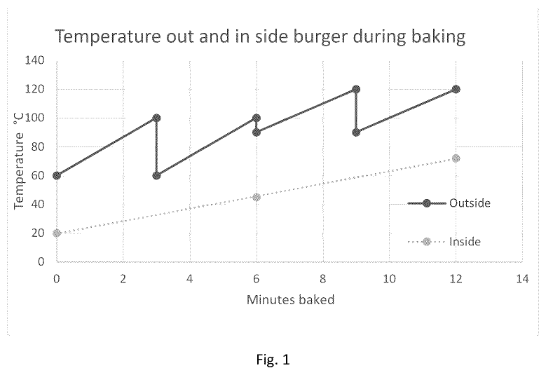

The present invention is further illustrated using the examples below, wherein FIG. 1 discloses the temperature profile during preparing a meat substitute hamburger.

### EXAMPLES

### Example 1

Meat substitute hamburgers with a composition as shown in table 1 were prepared as follows. Water was added to texturized soy and mixed until hydrated. Caramelized sugar, beet root powder, vitamins & minerals, salt and yeast extract where dry mixed separately and added to the hydrated soy. The remaining ingredients were added to the mixture which was subsequently hand mixed until homogenous appearance. The homogenous dough was chilled for 1 hour in the fridge. Hamburgers with a weight of 100 gram where subsequently shaped by hand. Subsequently the exterior of the hamburgers was coated with a flavour mix as shown in table 2. The hamburgers where frozen in the freezer for at least 3 nights before use.

TABLE 1

| Ingredient | Weight % |
|---|---|
| Soy texturized vegetable protein (ADM TU- | 15 |
| crumble caramel 180) | |
| Caramelized sugar (Buisman) | 0.1 |
| Beet root powder | 0.5 |
| High acyl gellan gum (DSM, Gellaneer ™ HD) | 0.5 |
| Methylcellulose | 1.5 |
| Sunflower oil | 10 |
| Soy protein concentrate (Myprotein) | 3 |
| Vitamines & minerals (B2, B3, B6, B12, iron, | 0.05 |
| selenium, zinc) | |
| Salt | 1 |
| Yeast extract (DSM, Maxarome ™ select) | 1.5 |
| Coconut fat | 4.5 |
| Water | 62.35 |

TABLE 2

| test | 0 | 1 | 2 | 3 | 4 | 5 | 6 | 7 |
|---|---|---|---|---|---|---|---|---|
| Ingredients (in | blanco | | | | | | | |
| grams) | | | | | | | | |
| Cysteine | | | | | | | 0.15 | 0.3 |
| Thiamine | | 0.3 | | 0.3 | | | 0.15 | 0.15 |
| Glucose | | | | 0.3 | | | | |
| Xylose | | | | | | | | 0.15 |
| Methionine | | 0.5 | 0.5 | 0.4 | | | 0.15 | 0.1 |
| Aromild ™⁻ (Kohjin) | | | | | | | | |
| Yeast extract | | 0.5 | 0.5 | 0.4 | 0.6 | | | |
| according to | | | | | | | | |
| WO2012163668 & | | | | | | | | |
| WO2007/073945 | | | | | | | | |
| (HG LSX) | | | | | | | | |
| Cystine | | | 0.45 | | | | | |
| Sunflower oil | | 1.5 | 1.5 | 1.5 | 1.5 | 1.4 | 1.5 | 1.5 |
| Maxavor Key Sulfur | | | | | 0.6 | | | |
| Yex-H (DSM Food | | | | | | | | |
| specialties) | | | | | | | | |
| Plantex MBE131 | | | | | | 0.6 | | |
| (DSM Food | | | | | | | | |
| specialties) | | | | | | | | |
| Total amount | 0 | 2.80 | 2.95 | 2.90 | 2.70 | 2.00 | 1.95 | 2.20 |

After defrosting the coated hamburger, they were fried in a pan having a temperature of 100° C. for 3 minutes per side, followed by 3 minutes per side with a pan temperature of 140° C. The temperature profile of the core temperature and exterior temperature is shown in FIG. 1.

### Results

A panel of 4 members assessed the appearance of the hamburgers after pan frying, the taste of the hamburgers and the general preference. The results are shown in table 3.

TABLE 3

| test | 0 | 1 | 2 | 3 | 4 | 5 | 6 | 7 |
|---|---|---|---|---|---|---|---|---|
| Appearance | Brown | Brown | Brown | Brown | Brown | Brown | Brown | Brown |
| | colour | colour | colour, | colour, | colour, | colour | colour | colour |
| | | | thicker | thicker | thicker | | | |
| | | | crust | crust | crust | | | |
| Taste | Soy | Meaty | Strongest | Good | Meaty | More | Weak | Weak |
| | veggie | impact, | and long | meaty, | impact, | roast and | meat | meat |
| | burger | less soy | lasting | slightly | less soy | slightly | impact | impact, |
| | like | aftertaste | meat | less | aftertaste | bitter | | soy |
| | | | taste | than 2 | | aftertaste | | aftertaste |
| Preference | − | ++ | ++++ | +++ | ++ | + | + | + |

The results show that coating the hamburgers results in a perceived meaty impact and a brown colour. Further, the results show that hamburgers coated with yeast extracts result in a thicker crust, a stronger meat impact and a reduction of soy aftertaste, which resulted in the highest score for the general preference.

1. A meat substitute provided with a flavour agent coating, optionally wherein the weight of the flavour agent coating is between 0.1% and 5% of the weight of the coated meat substitute.

2. The meat substitute according to claim 1, wherein the meat substitute is a meat substitute hamburger.

3. The meat substitute according to claim 1, wherein the flavour agent coating comprises one or more flavour precursors, optionally cysteine, thiamine, glucose, ribose, xylose, methionine and/or cystine.

4. The meat substitute according to claim 1, wherein the flavour agent coating comprises yeast extracts.

5. The meat substitute according to claim 4, wherein the yeast extracts comprise an amount of reducing sugar of 1% to 30% wt. of the yeast extract.

6. The meat substitute according to claim 4, wherein the yeast extracts comprise an amount of free asparagine, based on dry matter, which is not higher than 1 mg/g, optionally not higher than 0.2 mg/g, optionally not higher than 0.1 mg/g of the yeast extracts.

7. The meat substitute according to claim 4, wherein the yeast extract comprises an amount of gluten of less than 100 ppm, optionally less than 50 ppm gluten, optionally less than 40 ppm gluten, optionally less than 30 ppm gluten, based on salt free dry matter of the yeast extract.

8. The meat substitute according to claim 1, wherein the flavour agent coating comprises an oil, optionally a plant oil.

9. The meat substitute according to claim 1, wherein the meat substitute is a raw or uncooked meat substitute.

10. The meat substitute according to claim 2, wherein the hamburger comprises texturized vegetable protein, water, a binder and plant oil.

11. The meat substitute hamburger according to claim 2, wherein the hamburger comprises texturized soy protein.

12. A flavour agent coating composition comprising:
(i) 20% to 70% wt. of an oil;
(ii) 20% to 80% wt. of a yeast extract;
wherein the sum of (i) and (ii) does not exceed 100%.

13. The flavour agent coating composition according to claim 12, further comprising a reducing sugar and/or wherein the yeast extract comprises an amount of reducing sugar of 1% to 30% wt of the yeast extract and/or wherein the yeast extract comprises an amount of free asparagine, based on dry matter, which is not higher than 1 mg/g, optionally not higher than 0.2 mg/g, optionally not higher than 0.1 mg/g of the yeast extracts.

14. The flavour agent coating composition according to claim 1, wherein the yeast extract comprises an amount of gluten of less than 100 ppm, optionally less than 50 ppm gluten, optionally less than 40 ppm gluten, optionally less than 30 ppm gluten, based on salt free dry matter of the yeast extract.

15. A method for manufacturing a meat substitute as defined in claim 1, comprising coating a meat substitute with a flavour agent coating composition.
